(19) United States
(12) Patent Application Publication
(10) Pub. No.: US 2026/0354857 A1
(43) Pub. Date: Nov. 12, 2026

(54) ULTRASONIC TRANSDUCER

(71) Applicant: NAMI SURGICAL LIMITED, Glasgow (GB)

(72) Inventors: Nicola FENU, Glasgow (GB); Rebecca CLEARY, Glasgow (GB); Alexandr KIYASHKO, Glasgow (GB)

(21) Appl. No.: 19/491,666

(22) PCT Filed: Jun. 13, 2024
(86) PCT No.: PCT/GB2024/051510

§ 371 Date: Dec. 9, 2025

(30) Foreign Application Priority Data
Jun. 13, 2023 (GB) 2308810.7

(51) Int. Cl. A61B 17/32; A61B 17/00

(52) U.S. Cl. CPC A61B 17/320068; A61B 2017/00477; A61B 2017/320074; A61B 2017/320082

(57) ABSTRACT

An ultrasonic transducer for surgical applications is disclosed. The ultrasonic transducer comprises: a back mass; a front mass; an ultrasonic actuator arrangement held between the back mass and the front mass; an ultrasonic horn arrangement forward of the front mass; and a threaded fastening. The back mass, ultrasonic actuator arrangement, front mass and ultrasonic horn arrangement are arranged along a longitudinal axis of the transducer. The threaded fastening extends through the back mass and ultrasonic actuator arrangement, and couples to the front mass or to the ultrasonic horn arrangement. The ultrasonic transducer has improved energy transfer, and is cheaper and/or easier to assemble.

The present invention relates to an ultrasonic transducer and to a method of operation of an ultrasonic transducer, particularly, although not necessarily exclusively, for surgical applications.

### BACKGROUND TO THE INVENTION

Ultrasonic transducers are known in the art for a variety of applications. One such application is in ultrasonic surgical devices (either for hard or soft tissue), which adopt ultrasonic vibrations to enhance cutting performance, employing a transducer mounted in a hand-held device.

A prior art transducer is disclosed in FIGS. 29 and 30 of WO2023007013 and reproduced as present FIGS. 1A and 1B. FIG. 1A shows the transducer in exploded side rear side perspective view, and FIG. 1B shows the transducer in front perspective view with the ultrasonic horn arrangement displaced.

The transducer 100 has a back mass 101 and a front mass 102 (having a proximal portion 102a , intermediate portion 102b and distal portion 102c) with an ultrasonic horn arrangement 103 located forwards of the front mass. Two piezoceramic rings 107 of opposing polarity sandwich an electrode 106 to form a piezoelectric stack 108 (more broadly, an ultrasonic actuator arrangement) which is held between the back mass 101 and the front mass 102 by a prestressing bolt 104 and nut 105. The prestressing bolt 104 applies and distributes prestress within the piezoelectric stack 108. The front mass 102 has an annular portion which takes the form of an outer cylindrical wall circumscribing the longitudinal axis A. In operation, a driving signal is applied to electrode 106 and the front 102 and back 101 masses are earthed, causing oscillation of the piezoelectric rings 107. The back mass 101, piezoceramic rings 107, electrodes 106, the front mass 102 and ultrasonic horn arrangement 103 are arranged along a longitudinal axis A of the transducer. Vibrations generated by the piezoceramic rings 107 are conducted into the front mass 102 and into the ultrasonic horn arrangement 103 along a vibrational energy transfer path. The vibrations are then amplitude amplified by the ultrasonic horn arrangement 103.

However, there are some limitations associated with the configuration of this transducer (that is, the arrangement of the components of the transducer). Firstly, there are several metal interfaces within the transducer. If such an interface is not “tight”, this creates an air gap which behaves as a dielectric layer. The consequence is that the two components do not “see” each other to the extent desired, and results in an increase in undesirable energy dissipation. Secondly, the transducer is relatively difficult to assemble, in that it is challenging to fully tighten the horn onto the front mass. This is because there is a limited thickness on the ultrasonic horn arrangement (approximately 1 mm) for a tightening clamp to hold on to.

### SUMMARY OF THE INVENTION

There is generally a need for an apparatus and method which addresses one or more of the problems identified above.

It is an object of one or more aspects of the present invention to provide an ultrasonic transducer that reduces the number of metal interfaces within the transducer.

It is an object of one or more aspects of the present invention to provide an ultrasonic transducer that is easier to assemble than transducers known in the art.

Further aims and objects of the invention will become apparent from reading the following description.

According to a first aspect of the invention, there is provided an ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass; and
a threaded fastening,
   wherein the back mass, ultrasonic actuator arrangement, front mass and ultrasonic horn arrangement are arranged along a longitudinal axis of the transducer,
   wherein the threaded fastening extends through the back mass and ultrasonic actuator arrangement, and
   wherein the threaded fastening couples to the front mass or to the ultrasonic horn arrangement.

The inventors have found the ultrasonic transducers of the present invention to have improved energy transfer, and are cheaper and/or easier to assemble, compared to transducers known in the art.

By “couples” it is meant that the front mass or the ultrasonic horn arrangement is the counterpart to the threaded fastening. In other words, an end of the threaded fastening is located within the front mass or the ultrasonic horn arrangement.

Preferably, vibrations generated by the ultrasonic actuator arrangement are conducted into the front mass and into the ultrasonic horn arrangement along a vibrational energy transfer path and are amplitude amplified by the ultrasonic horn arrangement.

One or more of the back mass, front mass and ultrasonic horn arrangement may include a plurality of openings opening towards the longitudinal axis and intersecting the vibrational energy transfer path and configured to provide an increased mechanical compliance in a direction along the vibrational energy transfer path. Preferably, the front mass includes the plurality of openings. In some embodiments, the plurality of openings is not provided in the ultrasonic horn arrangement.

The front mass may comprise one or more regions having a plurality of openings and one or more regions absent of openings. The at least one region having a plurality of openings may be between a region absent of openings and the ultrasonic horn arrangement. The at least one region having a plurality of openings may be between two regions absent of openings. The regions preferably extend circumferentially.

The length of the transducer, measured from the proximal end of the back mass to the distal end of the ultrasonic horn arrangement, along the vibrational energy transfer path, preferably is not more than 40 mm.

The maximum diameter of the transducer, measured in a direction perpendicular to the length, preferably is not more than 15 mm.

The transducer may be a Langevin transducer. The ultrasonic actuator arrangement preferably comprises a piezoelectric stack. Preferably, the piezoelectric stack comprises an electrode and two piezoceramic rings of opposing polarity. The two piezoceramic rings sandwich the electrode.

The ultrasonic horn arrangement preferably comprises a blade, the blade preferably having a curved and/or tapered profile.

In some embodiments, the threaded fastening couples to the ultrasonic horn arrangement. In these embodiments, the threaded fastening preferably extends through the back mass, ultrasonic actuator arrangement and front mass and couples to the ultrasonic horn arrangement.

Advantages associated with these embodiments, when compared to the prior art transducer shown in FIG. 1, include, but are not limited to, one or more of higher Q factor; higher gain; and higher dynamic response from a low voltage. Additionally, these embodiments are easier to assemble than the prior art transducer shown in FIG. 1, at least partly because the component parts are less stiff to tighten.

The threaded fastening may be a bolt and may be termed a prestressing bolt. The prestressing bolt is used to apply and distribute prestress within the ultrasonic actuator arrangement. The prestressing bolt preferably comprises a threaded portion and a head.

In some embodiments, the head of the prestressing bolt is adjacent to the back mass.

The prestressing bolt may be a M3 bolt, preferably a M3×20 mm bolt.

The front mass may have an outer diameter of from about 3 mm to about 13 mm, or from about 4 mm to about 12 mm, or from about 5 mm to about 11 mm, or from about 6 mm to about 10 mm. The front mass may have an inner diameter of from about 1 mm to about 8 mm, or from about 1 mm to about 7 mm, or from about 2 mm to about 6 mm, or from about 3 mm to about 5 mm. This relatively large diameter advantageously permits for larger openings, when the front mass includes the plurality of openings.

The front mass may comprise one or more clamping slots. The purpose of slots is to improve ease of assembly of the transducer. The slots facilitate a tightening clamp to hold onto the front mass. The slots are preferably in a region of the front mass absent of openings. For the avoidance of doubt, the one or more clamping slots are distinct from the above-mentioned plurality of openings, serving a different purpose and providing a different technical effect.

In some embodiments, the threaded fastening couples to the front mass. In these embodiments, the front mass and ultrasonic horn arrangement preferably form a unitary structure. While the front mass and ultrasonic horn arrangement may be attached (e.g. welded) together, it is preferred that the front mass and ultrasonic horn arrangement are formed as a single structure (for example, through use of computer numerical control techniques).

The inventors of the present invention have found that, compared to transducers known in the art, not only is the ultrasonic transducer according to these embodiments cheaper to make, there are also fewer metal interfaces. This improves performance of the ultrasonic transducer, because having fewer interfaces results in fewer air gaps and lower undesirable energy dissipation.

Additionally, by forming the front mass and ultrasonic horn arrangement as a unitary structure, there is a larger mass available for a tightening clamp to hold onto, which facilitates easier assembly.

The front mass may comprise one or more regions having a plurality of openings and one or more regions absent of openings. Preferably, the threaded fastening couples with a region of the front mass absent of openings, wherein the region of the front mass absent of openings is adjacent to the ultrasonic actuator arrangement. This advantageously means that the region of the front mass having a plurality of openings is not prestressed by the bolt (i.e. the openings are not under compression and are unaffected by the stiffness of the threaded fastening).

The threaded fastening may be a bolt and may be termed a prestressing bolt. The prestressing bolt is used to apply and distribute prestress within the ultrasonic actuator arrangement. The prestressing bolt preferably comprises a threaded portion and a head.

In some embodiments, the head of the prestressing bolt is adjacent to the back mass.

The prestressing bolt may be a M3 bolt, preferably a M3×12 mm bolt.

The front mass may be tapered, preferably wherein the front mass tapers towards the ultrasonic horn arrangement. That is, the widest diameter of the front mass is in the region adjacent to the ultrasonic actuator arrangement, and the narrowest diameter of the front mass is in the region adjacent to the ultrasonic horn arrangement.

The front mass may comprise one or more slots. The purpose of slots is to improve ease of assembly of the transducer. The slots facilitate a tightening clamp to hold onto the front mass. The slots are preferably in a region of the front mass absent of openings.

Instead of a prestressing bolt (with a head and a single threaded portion), the threaded fastening may be a double ended bolt. Preferably, the double ended bolt comprises a central (preferably unthreaded) portion, a first threaded portion and a second threaded portion. The first threaded portion and second threaded portion extend in opposing directions from the central portion. Preferably, the first threaded portion has a first radius and the second threaded portion has a second radius, wherein the first radius is different to the second radius. In other words, the double ended bolt preferably comprises two threaded portions with different radii.

The first threaded portion preferably couples to the front mass. The second threaded portion preferably extends through the back mass and ultrasonic actuator arrangement.

Thus, when assembled, the central portion may preferably be positioned between the ultrasonic actuator arrangement and the front mass.

The second threaded portion may couple with a nut positioned adjacent to the back mass.

The central portion may have a diameter of from about 5 mm to about 11 mm, or from about 6 mm to about 10 mm, or from about 7 mm to about 9 mm, or about 8 mm. The central portion may have a diameter of at least about 5 mm. The central portion may have a diameter of no more than about 11 mm.

The first threaded portion may have a diameter of from about 3 mm to about 9 mm, or from about 4 mm to about 8 mm, or from about 5 mm to about 7 mm, or about 6 mm. The first threaded portion may have a diameter of at least about 3 mm. The first threaded portion may have a diameter of no more than about 9 mm.

The first threaded portion may have a length of from about 1 mm to about 6 mm, or from about 1 mm to about 5 mm, or from about 2 mm to about 4 mm, or about 3 mm. The first threaded portion may have a length of at least about 1 mm. The first threaded portion may have a length of no more than about 6 mm.

The first threaded portion may be a M6 thread, preferably a M6×3 mm thread.

The second threaded portion may have a diameter of from about 1 mm to about 6 mm, or from about 1 mm to about 5 mm, or from about 2 mm to about 4 mm, or about 3 mm. The second threaded portion may have a diameter of at least about 1 mm. The second threaded portion may have a diameter of no more than about 6 mm.

The second threaded portion may have a length of from about 7 mm to about 13 mm, or from about 8 mm to about 12 mm, or from about 9 mm to about 11 mm, or about 10 mm. The second threaded portion may have a length of at least about 7 mm. The second threaded portion may have a length of no more than about 13 mm.

The second threaded portion may be a M3 thread, preferably a M3×10 mm thread.

The front mass may have an outer diameter of from about 3 mm to about 11 mm, or from about 4 mm to about 10 mm, or from about 5 mm to about 9 mm, or from about 6 mm to about 8 mm. This relatively large diameter advantageously permits for larger openings, when the front mass includes the plurality of openings.

Preferably, the double ended bolt is made of, or at least comprises, titanium.

The central portion of the double ended bolt may comprise one or more slots, for receiving a tightening clamp.

The ultrasonic transducer may be for surgical, therapeutic, and/or diagnostic applications. For the avoidance of doubt, these applications include dentistry. The ultrasonic transducer may be for human and/or veterinary usage.

According to a second aspect of the invention, there is provided an ultrasonic transducer for surgical applications, the ultrasonic transducer comprising:
a back mass;
a front mass; a
n ultrasonic actuator arrangement held between the back mass and the front mass;
an ultrasonic horn arrangement forward of the front mass; and
a threaded fastening,
   wherein the back mass, ultrasonic actuator arrangement, front mass and ultrasonic horn arrangement are arranged along a longitudinal axis of the transducer, and
   wherein the threaded fastening extends through the back mass, ultrasonic actuator arrangement and front mass and couples to the ultrasonic horn arrangement.

Embodiments of the second aspect of the invention may include one or more features of the first aspect of the invention or their embodiments, or vice versa.

According to a third aspect of the invention, there is provided an ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass, wherein the front mass and ultrasonic horn arrangement form a unitary structure; and
a threaded fastening,
   wherein the back mass, ultrasonic actuator arrangement, front mass and ultrasonic horn arrangement are arranged along a longitudinal axis of the transducer,
   wherein the threaded fastening extends through the back mass and ultrasonic actuator arrangement, and
   wherein the threaded fastening couples to the front mass.

Embodiments of the third aspect of the invention may include one or more features of the first and second aspects of the invention or their embodiments, or vice versa.

According to a fourth aspect of the invention, there is provided an ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass, wherein the front mass and ultrasonic horn arrangement form a unitary structure; and
a prestressing bolt,
   wherein the back mass, ultrasonic actuator arrangement, front mass and ultrasonic horn arrangement are arranged along a longitudinal axis of the transducer,
   wherein the prestressing bolt extends through the back mass and ultrasonic actuator arrangement, and
   wherein the prestressing bolt couples to the front mass.

Embodiments of the fourth aspect of the invention may include one or more features of the first to third aspects of the invention or their embodiments, or vice versa.

According to a fifth aspect of the invention, there is provided an ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass, wherein the front mass and ultrasonic horn arrangement form a unitary structure; and
a double-ended bolt,
   wherein the back mass, ultrasonic actuator arrangement, front mass and ultrasonic horn arrangement are arranged along a longitudinal axis of the transducer,
   wherein the double-ended bolt extends through the back mass and ultrasonic actuator arrangement, and
   wherein the double-ended bolt couples to the front mass.

Embodiments of the fifth aspect of the invention may include one or more features of the first to fourth aspects of the invention or their embodiments, or vice versa.

According to a sixth aspect of the invention, there is provided a kit of parts comprising parts operable to be assembled into an ultrasonic transducer according to any one of the first to fifth aspects of the present invention.

Embodiments of the sixth aspect of the invention may include one or more features of the first to fifth aspects of the invention or their embodiments, or vice versa.

According to a seventh aspect of the invention, there is provided a surgical tool comprising an ultrasonic transducer according to any one of the first to fifth aspects of the present invention.

The surgical tool may further comprise one or more of a casing, a clamping jaw, and a mechanism for actuating the clamping jaw.

The surgical tool may be operated by a human or by a programmable machine, such as a robot.

Embodiments of the seventh aspect of the invention may include one or more features of the first to sixth aspects of the invention or their embodiments, or vice versa.

According to an eighth aspect of the invention, there is provided a method of operation of an ultrasonic transducer according to any one of the first to fifth aspects of the present invention.

The method preferably comprises applying an electrical signal to the ultrasonic actuator arrangement to generate vibrations to be conducted into the front mass and into the ultrasonic horn arrangement along a vibrational energy transfer path. Preferably the vibrations are amplitude amplified by the ultrasonic horn arrangement.

The ultrasonic transducer may be operated at a power density in the range of from 10 to 1000 W cm⁻². The ultrasonic transducer may be operated at a power in the range of from 1 to 1000 W.

Embodiments of the eighth aspect of the invention may include one or more features of the first to seventh aspects of the invention or their embodiments, or vice versa.

### BRIEF DESCRIPTION OF THE DRAWINGS

There will now be described, by way of example only, various embodiments of the invention with reference to the drawings, of which:

FIG. 1 shows a transducer of the prior art, in exploded side rear side perspective view (FIG. 1A), and in front perspective view (FIG. 1B);

FIG. 2 shows a transducer in accordance with an embodiment of the present invention, in side perspective view (FIG. 2A), in exploded side perspective view (FIG. 2B), in side view (FIG. 2C), and in sectional view along longitudinal axis A (FIG. 2D);

FIG. 3 shows a transducer in accordance with an embodiment of the present invention, in side perspective view (FIG. 3A), in exploded side perspective view (FIG. 3B), in side view (FIG. 3C), and in sectional view along longitudinal axis A (FIG. 3D); and

FIG. 4 shows a transducer in accordance with an embodiment of the present invention, in side perspective view (FIG. 4A), in exploded side perspective view (FIG. 4B), in side view (FIG. 4C), and in sectional view along longitudinal axis A (FIG. 4D).

### DETAILED DESCRIPTION OF THE PREFERRED EMBODIMENTS

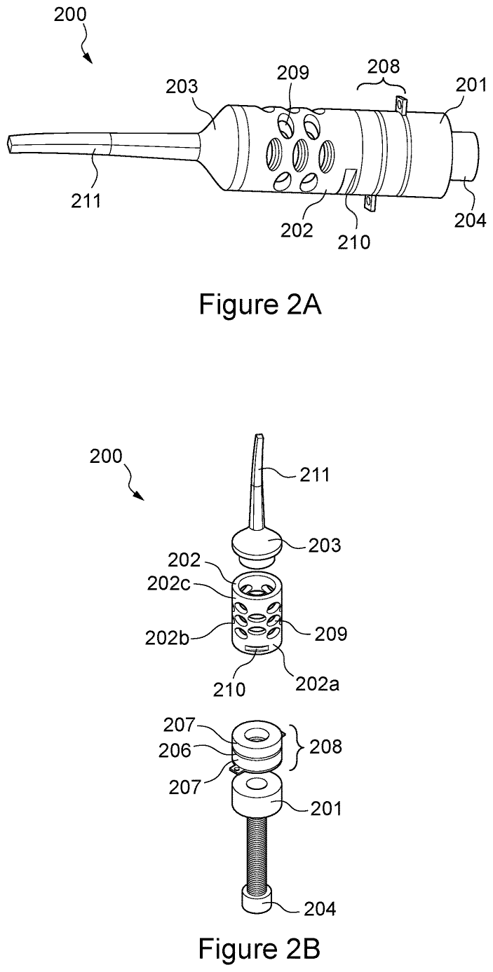

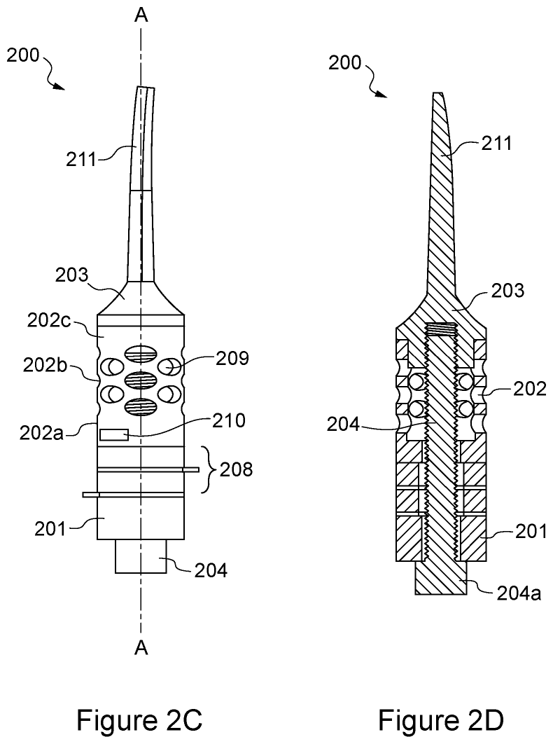

An explanation of the present invention will now be described with reference to FIGS. 2 to 4.

Referring to FIG. 2, there is shown generally at 200 a transducer according to an embodiment of the present invention. The transducer has a back mass 201 and a front mass 202 with an ultrasonic horn arrangement 203 located forwards of the front mass 202. Two piezoceramic rings 207 of opposing polarity sandwich an electrode 206 to form a piezoelectric stack 208 (i.e. an ultrasonic actuator arrangement). The back mass 201, piezoelectric stack 208, front mass 202 and ultrasonic horn arrangement 203 are arranged along a longitudinal axis A of the transducer 200.

The piezoelectric stack 208 is held between the back mass 201 and the front mass 202 by a threaded fastening which, in this particular embodiment, is a prestressing bolt 204. In this embodiment, the prestressing bolt 204 is a M3×20 mm bolt. However, it will be appreciated that the type of bolt is not limited as such and will be dependent on the desired size of the transducer. The head of the bolt 204a is positioned adjacent to the back mass 201. The threaded portion of the prestressing bolt 204 extends through the back mass 201, piezoelectric stack 208 and front mass 202 and couples to the ultrasonic horn arrangement 203.

In this embodiment, the front mass 202 has an arrangement of openings (i.e. holes) 209 formed through it. The openings open towards the longitudinal axis A and intersect the vibrational energy transfer path, providing an increased mechanical compliance in an axial direction parallel to the longitudinal axis A and along the vibrational energy path.

The front mass 202 has a proximal portion 202a, intermediate portion 202b and distal portion 202c. The proximal portion 202a is in contact with the piezoelectric stack 208. The threaded portion of the prestressing bolt 204 couples to the proximal portion 202a of the front mass 202. The openings 209 are formed in the intermediate portion 202b. The proximal portion 202a and distal portion 202c are absent of any openings. Instead, the proximal portion 202a has one or more slots 210. The purpose of slots 210 is to improve ease of assembly of the transducer 200. The slots 210 facilitate a tightening clamp to hold onto the front mass 202.

The distal portion 202c is in contact with the ultrasonic horn arrangement 203. The proximal portion 202a, intermediate portion 202b and distal portion 202c of the front mass 202 are formed integrally with each other and have substantially the same outer diameter (ignoring the effect of the presence of the openings 209 on the outer diameter).

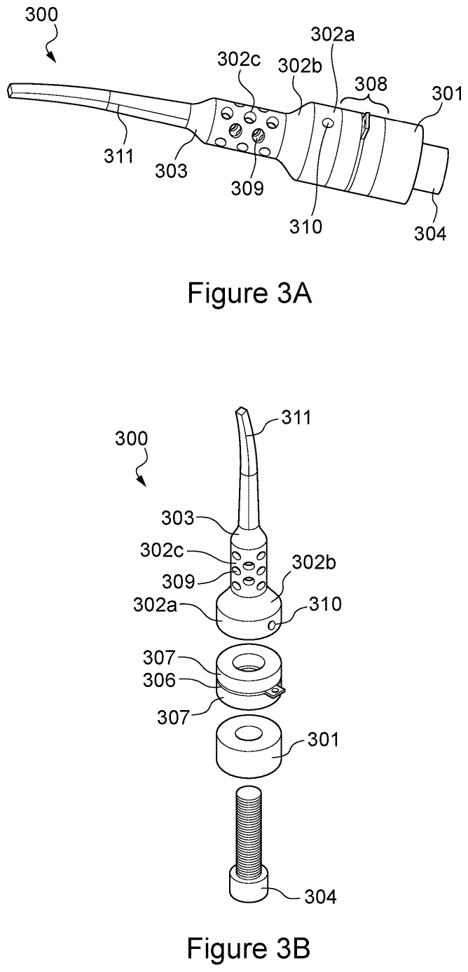

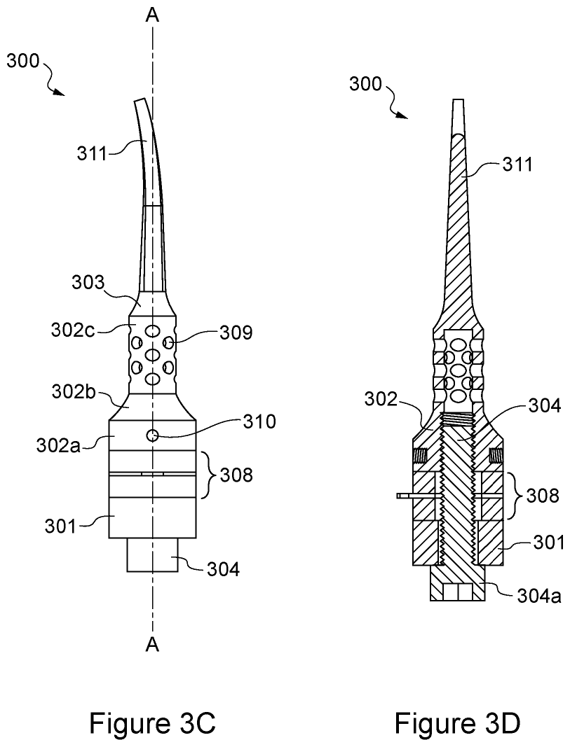

Referring to FIG. 3, there is shown generally at 300 a transducer according to an embodiment of the present invention. The transducer has a back mass 301 and a front mass 302 with an ultrasonic horn arrangement 303 located forwards of the front mass 302. Two piezoceramic rings 307 of opposing polarity sandwich an electrode 306 to form a piezoelectric stack 308 (i.e. an ultrasonic actuator arrangement). The back mass 301, piezoelectric stack 308, front mass 302 and ultrasonic horn arrangement 303 are arranged along a longitudinal axis A of the transducer 300. The front mass 302 and ultrasonic horn arrangement 303 together form a unitary structure. In this embodiment, the front mass 302 and ultrasonic horn arrangement 303 are made as a single component (that is, they are not made separately and subsequently attached).

The piezoelectric stack 308 is held between the back mass 301 and the front mass 302 by a threaded fastening which, in this particular embodiment, is a prestressing bolt 304. In this embodiment, the prestressing bolt 304 is a M3×12 mm bolt. However, it will be appreciated that the type of bolt is not limited as such and will be dependent on the desired size of the transducer. The head of the bolt 304a is positioned adjacent to the back mass 301. The threaded portion of the prestressing bolt 304 extends through the back mass 301 and piezoelectric stack 308, and couples to the front mass 302.

In this embodiment, the front mass 302 has an arrangement of openings (i.e. holes) 309 formed through it. The openings open towards the longitudinal axis A and intersect the vibrational energy transfer path, providing an increased mechanical compliance in an axial direction parallel to the longitudinal axis A and along the vibrational energy path.

The front mass 302 has a proximal portion 302a, intermediate portion 302b and distal portion 302c. The proximal portion 302a is in contact with the piezoelectric stack 308. The threaded portion of the prestressing bolt 304 couples to the intermediate portion 302b of the front mass 302. The openings 309 are formed in the distal portion 302c. The proximal portion 302a and intermediate portion 302b are absent of any openings. Instead, the proximal portion 302a has one or more slots 310. The purpose of slots 310 is to improve ease of assembly of the transducer 300. The slots 310 facilitate a tightening clamp to hold onto the front mass 302.

The distal portion 302c is in contact with the ultrasonic horn arrangement 303. The proximal portion 302a, intermediate portion 302b and distal portion 302c of the front mass 302 are formed integrally with each other.

The diameter of the proximal portion 302a is greater than the diameter of the distal portion 302c. The intermediate portion 302b has a tapered profile such that its widest diameter is equal to the diameter of the proximal portion 302a and its narrowest diameter is equal to the diameter of the distal portion 302c. Thus, the front mass 302 can be said to be a tapered front mass.

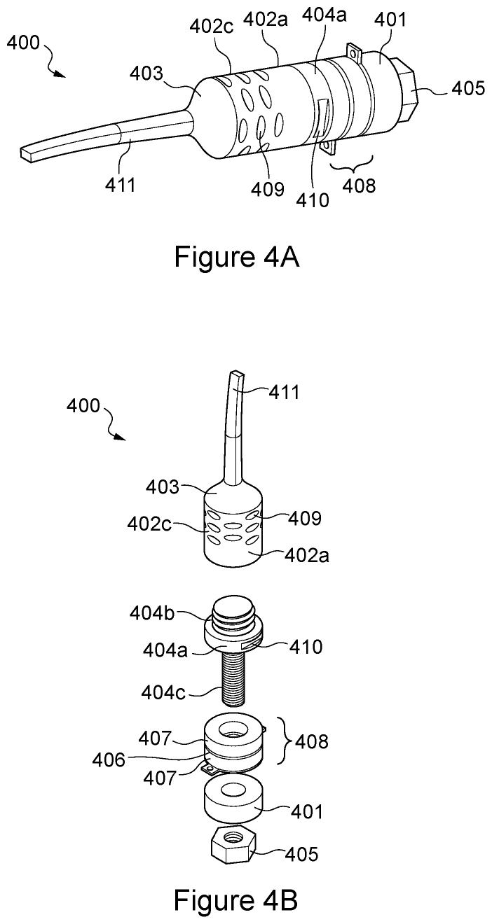

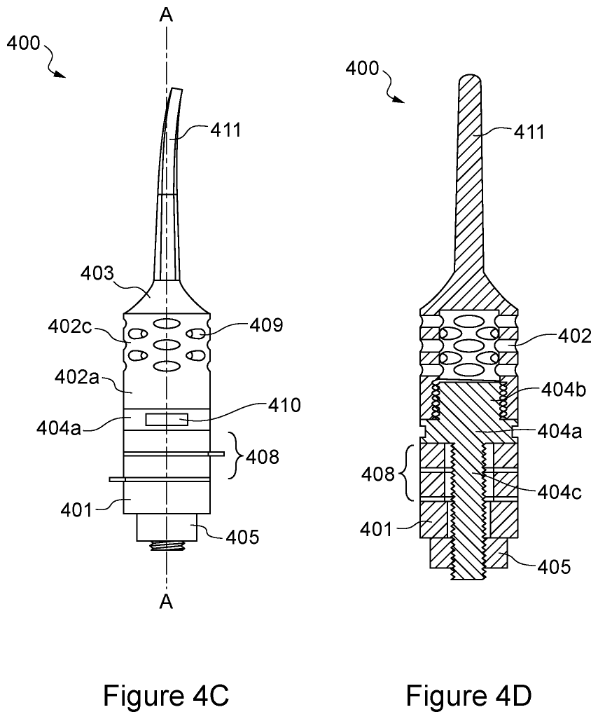

Referring to FIG. 4, there is shown generally at 400 a transducer according to an embodiment of the present invention.

The transducer has a back mass 401 and a front mass 402 with an ultrasonic horn arrangement 403 located forwards of the front mass 402. Two piezoceramic rings 407 of opposing polarity sandwich an electrode 406 to form a piezoelectric stack 408 (i.e. an ultrasonic actuator arrangement). The back mass 401, piezoelectric stack 408, front mass 402 and ultrasonic horn arrangement 403 are arranged along a longitudinal axis A of the transducer 400. The front mass 402 and ultrasonic horn arrangement 403 together form a unitary structure. In this embodiment, the front mass 402 and ultrasonic horn arrangement 403 are made as a single component (that is, they are not made separately and subsequently attached).

The piezoelectric stack 408 is held between the back mass 401 and the front mass 402 by a threaded fastening which, in this particular embodiment, is a double ended bolt 404. The double ended bolt 404 has a central portion 404a and two threaded portions: a first threaded portion 404b and a second threaded portion 404c. The two threaded portions 404b, 404c are on adjacent faces of the central portion 404a and extend in opposing directions. The diameter of the central portion 404a is greater than the diameter of the first threaded portions 404b, which is greater than the diameter of the second threaded portion 404c.

The first threaded portion 404b couples to the front mass 402. The second threaded portion 404c extends through the back mass 401 and piezoelectric stack 408 and couples with a nut 405 positioned adjacent to the back mass 401 (i.e. on the face of the back mass 401 opposite the face which is adjacent to the piezoelectric stack 408). The inner diameter of the nut 405 is selected to be commensurate with the outer diameter of the threaded portion 404c in a matter which is known in the art. The second threaded portion 404c applies prestress to the piezoelectric stack 408.

In this embodiment, the front mass 402 has an arrangement of openings (i.e. holes) 409 formed through it. The openings open towards the longitudinal axis A and intersect the vibrational energy transfer path, providing an increased mechanical compliance in an axial direction parallel to the longitudinal axis A and along the vibrational energy path.

The front mass 402 has a proximal portion 402a and distal portion 402c. The proximal portion 402a is in contact with the piezoelectric stack 408. The openings 409 are formed in the distal portion 402c. The distal portion 402c is in contact with the ultrasonic horn arrangement 403. The proximal portion 402a and distal portion 402c of the front mass 402 are formed integrally with each other and have substantially the same outer diameter (ignoring the effect of the presence of the openings 409 on the outer diameter).

The first threaded portion 404b couples to the proximal portion 402a of the front mass 402. This means that the bolt 404 does not couple to the region of the front mass 402 having openings 409, and thus does not interfere with the advantageous technical effects imparted by the presence of the openings 409.

The central portion 404a of the double ended bolt 404 has one or more slots 410. The purpose of slots 410 is to improve ease of assembly of the transducer 400. The slots 410 facilitate a clamp to hold onto the central portion 404a, and allow for the nut 405 and ultrasonic horn arrangement 403 to be screwed on to the double ended bolt 404.

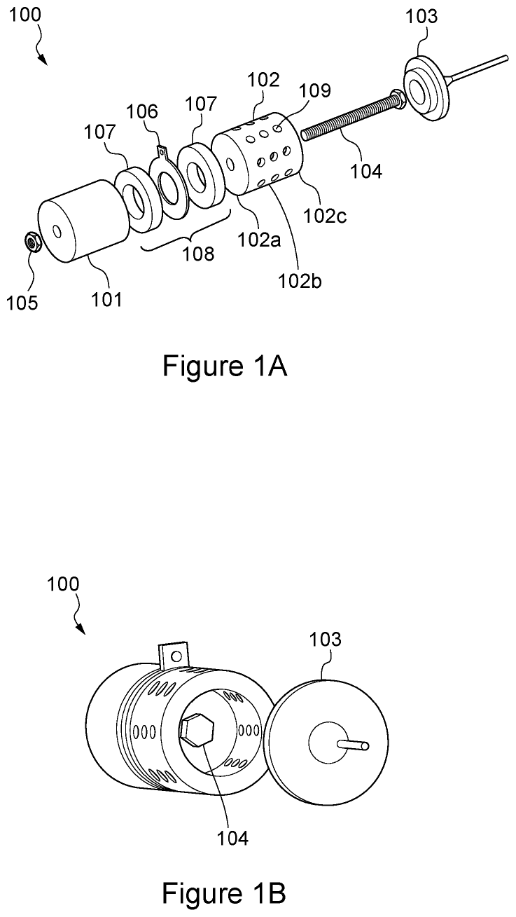

Additionally, the entirety of the ultrasonic horn arrangement 403 can advantageously be held by a tightening clamp during screwing. This is compared to the prior art transducer (in FIGS. 1 and 2), in which only a very small portion (approximately 1 mm) of the horn 103 can be held.

In each of the embodiments, the ultrasonic horn arrangement 203, 303, 403 comprises a blade 211, 311, 411, which is shaped according to the desired effect/application. In these embodiments, the blade 211, 311, 411 has a curved and tapered profile, such that the cross-sectional area at the distal end (i.e. distal to the front mass 202, 302, 402) is greater than the cross-sectional area at the proximal end.

Each of the embodiments operate in a way similar to the prior art transducer shown in FIG. 1. An electrical driving signal is applied to electrode 206, 306, 406 and the front 202, 302, 402 and back 201, 301, 401 masses are earthed, causing oscillation of the piezoelectric rings 207, 307, 407. Vibrations generated by the piezoceramic rings 207, 307, 407 are conducted into the front mass 202, 302, 402 and into the ultrasonic horn arrangement 203, 303, 403 along a vibrational energy transfer path. The vibrations are then amplitude amplified by the ultrasonic horn arrangement 203, 303, 403.

Each of the embodiments can be used in a surgical tool. In addition to the ultrasonic transducer, the surgical tool may comprise, for example, one or more of a casing, a clamping jaw, and a mechanism for actuating the clamping jaw.

An ultrasonic transducer 200, 300, 400 for surgical applications is disclosed. The ultrasonic transducer comprises: a back mass 201, 301, 401; a front mass 202, 302, 402; an ultrasonic actuator arrangement 208, 308, 408 held between the back mass and the front mass; an ultrasonic horn arrangement 203, 303, 403 forward of the front mass; and a threaded fastening 204, 304, 404. The back mass, ultrasonic actuator arrangement, front mass and ultrasonic horn arrangement are arranged along a longitudinal axis of the transducer. The threaded fastening extends through the back mass and ultrasonic actuator arrangement, and couples to the front mass or to the ultrasonic horn arrangement. The ultrasonic transducer has improved energy transfer, and is cheaper and/or easier to assemble.

Throughout the specification, unless the context demands otherwise, the terms “comprise”or “include”, or variations such as “comprises” or “comprising”, “includes” or “including” will be understood to imply the inclusion of a stated integer or group of integers, but not the exclusion of any other integer or group of integers. Furthermore, unless the context clearly demands otherwise, the term “or” will be interpreted as being inclusive not exclusive.

The foregoing description of the invention has been presented for purposes of illustration and description and is not intended to be exhaustive or to limit the invention to the precise form disclosed. The described embodiments were chosen and described in order to best explain the principles of the invention and its practical application to thereby enable others skilled in the art to best utilise the invention in various embodiments and with various modifications as are suited to the particular use contemplated. Therefore, further modifications or improvements may be incorporated without departing from the scope of the invention as defined by the appended claims.

1. An ultrasonic transducer for surgical applications, the ultrasonic transducer comprising:
a back mass;
a front mass;
an ultrasonic actuator arrangement held between the back mass and the front mass;
an ultrasonic horn arrangement forward of the front mass; and
a threaded fastening,
wherein the back mass, ultrasonic actuator arrangement, front mass and ultrasonic horn arrangement are arranged along a longitudinal axis of the transducer,
wherein the threaded fastening extends through the back mass and ultrasonic actuator arrangement, and
wherein the threaded fastening couples to the front mass or to the ultrasonic horn arrangement.

2. The ultrasonic transducer according to claim 1, wherein one or more of the back mass, front mass and ultrasonic horn arrangement includes a plurality of openings opening towards the longitudinal axis and intersecting a vibrational energy transfer path and configured to provide an increased mechanical compliance in a direction along the vibrational energy transfer path.

3. The ultrasonic transducer according to claim 1 wherein the front mass includes a plurality of openings.

4. (canceled)

5. The ultrasonic transducer according to claim 1, wherein the ultrasonic horn arrangement comprises a blade having a curved and/or profile, a tapered profile, or both a curved profile and a tapered profile.

6. The ultrasonic transducer according to claim 1, wherein the threaded fastening extends through the front mass and couples to the ultrasonic horn arrangement.

7. The ultrasonic transducer according to claim 6, wherein the threaded fastening extends through the back mass, ultrasonic actuator arrangement and front mass.

8. The ultrasonic transducer according to claim 7, wherein the threaded fastening is a prestressing bolt comprising a threaded portion and a head, and the head of the prestressing bolt is adjacent to the back mass.

9. (canceled)

10. The ultrasonic transducer according to claim 1, wherein the threaded fastening couples to the front mass.

11. The ultrasonic transducer according to claim 10, wherein the front mass and ultrasonic horn arrangement form a unitary structure.

12. The ultrasonic transducer according to claim 10, wherein the threaded fastening is a prestressing bolt.

13. The ultrasonic transducer according to claim 12, wherein the prestressing bolt comprises a threaded portion and a head, and the head of the prestressing bolt is adjacent to the back mass.

14. The ultrasonic transducer according to claim 12, wherein the front mass tapers towards the ultrasonic horn arrangement.

15. The ultrasonic transducer according to claim 10, wherein the threaded fastening is a double ended bolt comprising two threaded portions with different radii.

16. The ultrasonic transducer according to claim 15, wherein the double ended bolt comprises a central portion, a first threaded portion and a second threaded portion.

17. The ultrasonic transducer according to claim 16, wherein the first threaded portion couples to the front mass, and the second threaded portion extends through the back mass and ultrasonic actuator arrangement.

18. The ultrasonic transducer according to claim 17, wherein the second threaded portion couples with a nut positioned adjacent to the back mass.

19. A kit of parts comprising parts operable to be assembled into an ultrasonic transducer according to claim 1.

20. A surgical tool comprising an ultrasonic transducer according to claim 1.

21. A method of operation of an ultrasonic transducer according to any claim 1, the method comprising applying an electrical signal to the ultrasonic actuator arrangement to generate vibrations to be conducted into the front mass and into the ultrasonic horn arrangement along a vibrational energy transfer path.

22. The ultrasonic transducer of claim 1, wherein the front mass comprises one or more slots for receiving a tightening clap during assembly of the ultrasonic transducer.
